(19) United States
(12) Patent Application Publication
(10) Pub. No.: US 2002/0022804 A1
(43) Pub. Date: Feb. 21, 2002

(54) Syringe device

(76) Inventors: Eric Connolly, Milton Keynes (GB); Timothy Paul Hewlett, London (GB); William John Maskell, London (GB); Gerard Michael O'Brien, London (GB)

Correspondence Address: NIXON & VANDERHYE P.C., 8th Floor, 1100 North Glebe Road, Arlington, VA, 22201, US

(21) Appl. No.: 09/948,704

(22) Filed: Sep. 10, 2001

Related U.S. Application Data
(63) Continuation of application No. PCT/EP00/01994, filed on Mar. 8, 2000.

(30) Foreign Application Priority Data
Mar. 10, 1999 (GB) 9905326.6

(51) Int. Cl. A61M 5/24

(52) U.S. Cl. 604/201; 604/200

(57) ABSTRACT

There is provided a syringe comprising a barrel having inner walls defining a compartment in the barrel, a closable nozzle at the base of the barrel and a plunger movable longitudinally within the barrel compartment. The plunger has inner walls defining a chamber suitable to receive a vial. A hollow needle extends through the plunger to enable fluid communication between the plunger chamber and barrel compartment. A compressible and puncturable sealing element within the plunger chamber provides a sterile seal for the needle and barrel compartment.

This invention relates to disposable syringes, more particularly to disposable syringes of the type suitable for mixing two fluid components, in situ, before administration.

Syringes suitable for mixing two-component medicaments of the prior art comprise a barrel split into two compartments by a plunger portion, each compartment housing a pre-loaded medicament. Relative movement of the plunger portion within the barrel opens a bypass channel or dislodges a valve to enable fluid communication between the two compartments. Examples of such mechanisms are described in European Patent Application EP295337 and International Patent Application WO84/01291. Such devices are generally considered not to facilitate effective mixing of the two components.

Alternatively, other syringe devices employ a barrel portion having two compartments housing the medicaments, separated by two moveable sealable elements, between which is located a moveable stopper holding a double ended needle, and a plunger adapted to move within the barrel. As the plunger moves within the barrel, the sealable elements are constructed to be sequentially pierced by the double ended needle and mixing is effected by the stopper carrying the needle being forced away from the base of the barrel as liquid is forced into the first compartment. Such devices are described in UK Patent Application GB2002241 and International Patent Application WO94/28964.

One disadvantage of the syringes of the prior art is that they require pre-loading of both components in the syringe, under sterile conditions. Therefore, an active medicinal product must be packaged into such a syringe at source by the manufacturer. The present invention addresses this problem by presenting a syringe which requires pre-loading of a liquid diluent only, thus facilitating a more convenient and simplified packaging manufacture.

A further disadvantage of the syringes of the prior art is their lack of versatility, i.e. such a syringe may not be used for any medical product other than that which is packed therein. The device of the present invention, however, may receive a standard vial, with a piercable end, containing the medicinal product, and therefore the standard packaging process for such medicinal products does not require any modification. Further, since the device need incorporate only the liquid diluent, where this excipient is a versatile diluent, such as water, the medicinal product may be chosen from a number of suitable alternatives. Thus, the device of the present invention is much more versatile in use in a medical environment than those of the prior art.

Yet further, the syringes of the prior art incorporate a number of moving parts and a relatively complicated mechanism of action. The device of the present invention is of a much simpler design, incorporating less parts and is simpler to use than the devices of the prior art.

According to one aspect of the present invention there is provided a syringe comprising:

a barrel having inner walls defining a compartment in the barrel;

a closable nozzle at the base of the barrel;

a plunger movable longitudinally within said barrel compartment;

said plunger having inner walls defining a chamber suitable to receive a vial;

a hollow needle extending through the plunger to enable fluid communication between said plunger chamber and the barrel compartment; and

a compressible and puncturable sealing element within the plunger chamber to provide a sterile seal for the needle and the barrel compartment.

In use, a vial having a piercable end and containing a medicinal product is seated on the sealing element, a longitudinal force is applied to the vial to cause piercing of both the sealing element and the end of the vial by the hollow needle, thereby facilitating fluid communication between the vial and a liquid diluent which is pre-disposed in the barrel compartment;

longitudinal movement of the plunger within the barrel towards the closable nozzle creates a pressure gradient to force the liquid diluent into the vial, whereby the diluent and medicinal product are mixed;

removal of the force on the plunger causes a relative reverse pressure gradient to expel the diluted product from the vial and into the barrel compartment; and

then opening of the closable nozzle and renewed longitudinal movement of the plunger causes expellation of the diluted product through the closable nozzle in a conventional manner.

Preferably the base of the syringe barrel is provided with a projection extending beyond the end of the closable nozzle, to enable the syringe to be disposed in an upright position for mixing. This projection is suitably an elongate rim around the circumference of the base of the syringe, which is most conveniently unitary with the syringe body.

Suitably, a syringe of the present invention incorporates a liquid diluent in the barrel compartment. Preferably, the liquid diluent is sterilised water. Optionally, the diluent may contain conventional excipients such as pharmaceutically acceptable salts or buffers and/or further active ingredients.

Alternatively, the barrel compartment of the syringe contains a medicinal product.

Preferably a syringe according to the present invention incorporates a vial of a conventional design, having a piercable end. Preferably the vial contains a medicinal product. It will be clear that although the vial is typically manufactured from glass, in the context of the present invention, a vial may be any suitably constructed vessel to carry a medicinal product and having a piercable seal.

Preferably the medicinal product is a drug in powder form. The medicinal product may be characteristically unstable when solubilised or suspended in water over a long period of time. The invention is particularly suitable for use with an injectable cephalosporin.

As a further aspect of the present invention, the syringe additionally comprises reversible retention means to retain the vial inside the plunger chamber, after piercing by the hollow needle, to allow the operative to release the force on the vial.

Preferably the reversible retention means is attachable to the inner wall of the plunger chamber. Alternatively the reversible retention means is mouldable into the inner wall of the plunger chamber.

Suitably the reversible retention means comprises clip beads.

Following mixing and dispensing of the drug, removal of the vial is optional. Removal of the vial may be achieved by removing the restriction imposed on the vial by the retention means. Any suitable method for removal of the vial may be used, for instance, by either distortion of the plunger chamber which causes clip beads to move away from the vial, distortion of localised areas of the plunger chamber which causes only clip beads to move, rotary or vertical movement of the secondary component which allows the clip beads to move, or a frangible section on the plunger chamber which negates the effect of the clip beads.

Alternatively, the vial is unrestrained within the device, such that it may be removed, for instance after the diluent is forced into the vial, to enable vigorous mixing of the vial contents by the operative.

As a further aspect of the present invention, the syringe may comprise a second reversible retention means to retain the plunger at, or substantially next to, the base of the barrel, to enable the operative to release the force on the plunger and commence a more vigorous mixing action.

Suitably, the plunger retention means comprises a ratchet mechanism which prevents the plunger from returning until it is acted upon in such a way as to negate the ratchet mechanism.

Alternatively the plunger retention means comprise a latching mechanism which retains the plunger at or near the base of the chamber.

Plunger retention can be achieved automatically upon depression of the plunger, or can be induced by horizontal or rotary movement of the plunger. The latching can also be achieved by moving part of the barrel within which the plunger is operating to achieve the same effect. Release of the plunger is achieved by distortion of localised areas of the barrel which causes only the latching section to move, or by rotary or horizontal movement of the plunger to bypass the latching portion of the barrel moulding. In the case of the ratchet mechanism it may be necessary to further introduce the plunger into the barrel causing the ratchet sections of the barrel or plunger to be changed such that they negate the ratchet effect from that point onwards, akin to a light bulb bayonet mechanism.

Suitably, the sealing element is a substantially cylindrical rubber diaphragm, which adheres to the base of the plunger and forms a sleeve around the hollow needle. The sealing element may be adhered to the plunger by any suitable means. It is preferable that if the vial containing the mixed product is removed from the syringe during the mixing process, the sealing element re-isolates the needle and the barrel compartment from the external environment.

Preferably the hollow needle comprises a metal. Preferably the metal is steel.

In order to provide a more stable support for the needle, the plunger may have a resilient rubber base through which the needle forms the conduit.

Preferably the closable nozzle comprises a luer taper cap.

The syringe according to the present invention is suitable for use for dispensing medicinal product.

As a preferred embodiment, the invention is hereby described with reference to the following non-limiting example wherein:

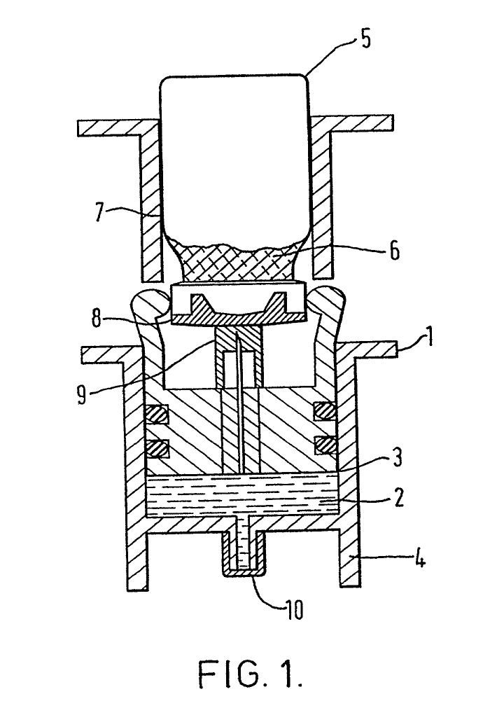

FIG. 1 represents a syringe according to the present invention with a vial containing powdered drug substance seated on a sealing diaphragm;

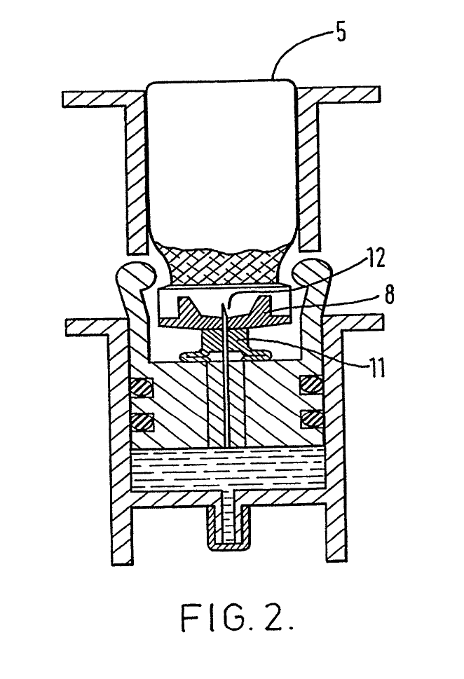

FIG. 2 represents the syringe where the vial has been forced down on the diaphragm and both the diaphragm and vial seal have been pierced by the hollow needle;

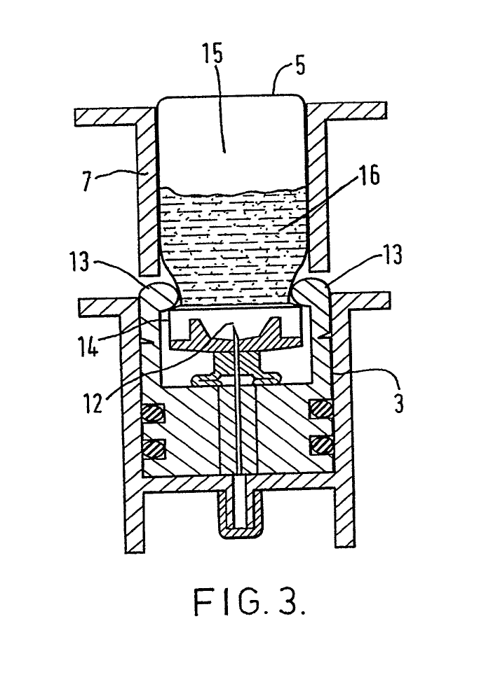

FIG. 3 represents the infusion step, whereby the diluent and medicament are mixed by the pressure gradient formed in the device;

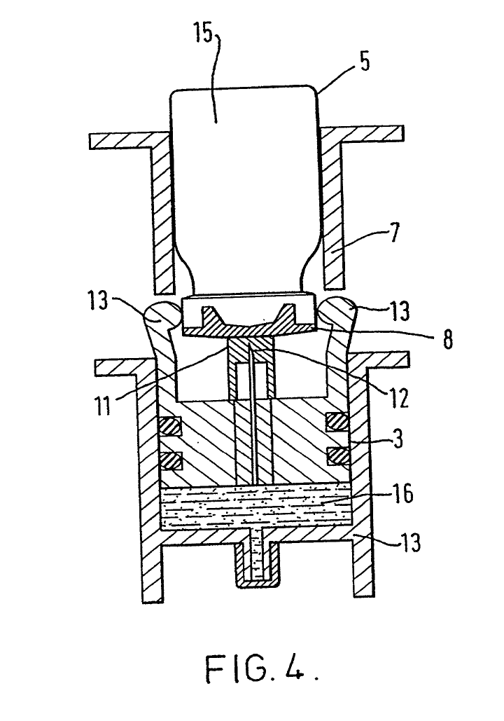

FIG. 4 represents the release step, whereby the force on the plunger holding the vial is removed so that it raises automatically and the diluted medicament is expelled into the barrel; and

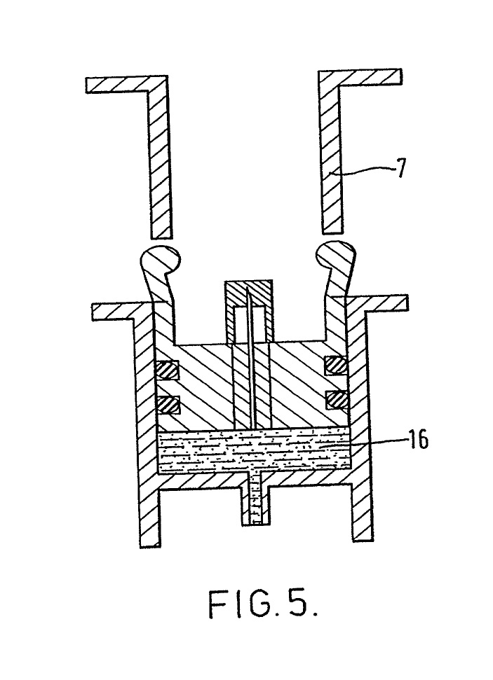

FIG. 5 represents the syringe, ready to dispense the diluted product.

With reference to the FIG. 1, the syringe 1 is pre-loaded with the correct volume of a liquid diluent 2, normally water, in the barrel 3, and may be tamper-evident sealed (not shown) to ensure sterility. When needed for dispensation, it is placed upright on a flat surface on a base projection 4, and a vial 5 containing a medicinal product 6 is inserted neck down into the plunger 7, so that the vial rubber seal 8 sits squarely on the rubber needle sleeve 9. The syringe is closed by means of a luer tap 10.

In FIG. 2, pressing down on the base of the vial 5 squashes the rubber needle sleeve 11 flat, thus forcing the needle 12 through the vial seal 8 and into the vial 5.

As shown in FIG. 3, continued downward force on the base of the vial 5 forces the plunger 7 down into the barrel 3 and the diluent up through the needle 12 into the vial 5 in a ‘fountain’ effect to mix with and dilute the medicinal product 16. As the plunger 7 completes its travel, jaws 13 come in on each side of the vial neck 14, and hold it fast, thus retaining a face-to-face seal. The operator can then cease pressing on the base of the vial 5. Further jaws (not shown) may also come into effect at the same time to secure the plunger at the base of the barrel.

At this stage the operator may shake the syringe to aid mixing. The head space 15 in the vial is pressurised at this time.

As shown in FIG. 4, when the drug is fully mixed, release of the force which holds the plunger 7 at the base of the barrel 3 causes the pressurised head space 15 in the vial to force the diluted product 16 back down the needle 12, and into the barrel 3. This raises the plunger 7 inside the barrel, and at the top of its travel, the jaws 13 around the neck of the vial 5 release.

The squashed needle sleeve 11 now returns to its original form, pushing the empty vial 5 upwards, withdrawing the needle 12 from the vial seal 8 and sealing the point of the needle 12.

According to FIG. 5, the loose vial can be removed from the plunger 7, the luer taper cap removed, and the diluted product 16 may be dispensed in a conventional manner. Both the syringe and vial may then be disposed of.

1. A syringe comprising:
a barrel having inner walls defining a compartment in the barrel;
a closable nozzle at the base of the barrel;
a plunger movable longitudinally within said barrel compartment;
said plunger having inner walls defining a chamber suitable to receive a vial;
a hollow needle extending through the plunger to enable fluid communication between said plunger chamber and the barrel compartment; and
a compressible and puncturable sealing element within the plunger chamber to provide a sterile seal for the needle and the barrel compartment.

2. A syringe according to claim 1 wherein the base of the barrel is provided with a projection extending beyond the end of the closable nozzle.

3. A syringe according to claim 2 wherein said projection is an elongate rim around the circumference of the base of the syringe.

4. A syringe according to claim 3 wherein said elongate rim is unitary with the syringe body.

5. A syringe according to any of claims 1 to 4 wherein the sealing element is a substantially cylindrical rubber diaphragm which adheres to the base of the plunger and forms a sleeve around the hollow needle.

6. A syringe according to any of claims 1 to 5 wherein the hollow needle comprises a metal.

7. A syringe according to claim 6 wherein the metal is steel.

8. A syringe according to any of claims 1 to 7 wherein the closable nozzle comprises a luer taper cap.

9. A syringe according to any of claims 1 to 8 additionally comprising reversible retention means to retain the plunger at or substantially next to the base of the barrel.

10. A syringe according to claim 9 wherein said reversible retention means comprise a ratchet mechanism.

11. A syringe according to claim 9 wherein said reversible retention means comprise a latching mechanism.

12. A syringe according to any of claims 1 to 11 additionally comprising a vial having a piercable end.

13. A syringe according to claim 12 additionally comprising a second reversible retention means to retain the vial inside the plunger chamber after piercing.

14. A syringe according to claim 13 wherein the second reversible retention means is attachable to the inner wall of the plunger chamber.

15. A syringe according to either of claims 13 wherein the second reversible retention means is mouldable into the inner wall of the plunger chamber.

16. A syringe according to any of claims 13 to 15 wherein the second reversible retention means comprises clip beads.

17. A syringe according to any of claims 1 to 16 wherein the barrel compartment contains a liquid diluent.

18. A syringe according to claim 17 wherein said liquid diluent is sterilised water.

19. A syringe according to either of claims 17 and 18 wherein the liquid diluent contains an excipient.

20. A syringe according to claim 19 wherein said excipient comprises a pharmaceutically acceptable salt or buffer.

21. A syringe according to any of claims 1 to 20 wherein the barrel compartment contains a medicinal product.

22. A syringe according to any of claims 12 to 20 wherein the vial contains a medicinal product.

23. A syringe according to either of claims 21 and 22 wherein the medicinal product is a drug in powder form.

24. A syringe according to claim 23 wherein the drug is cephalosporin.

25. Use of a syringe according to any of claims 1 to 24 for dispensing medicinal product.

26. Kit of parts comprising a syringe according to any of claims I to 11 and a vial having a piercable end.

27. Kit of parts according to claim 26 wherein the syringe contains liquid diluent.

28. Kit of parts according to either of claims 26 and 27 wherein the vial contains a medicinal product.
